(19) United States
(12) Patent Application Publication
(10) Pub. No.: US 2024/0024119 A1
(43) Pub. Date: Jan. 25, 2024

(54) FIXATION MECHANISM FOR GLENOID IMPLANTS

(71) Applicant: HOWMEDICA OSTEONICS CORP., Mahwah, NJ (US)

(72) Inventors: Michael Q. FREEHILL, Edina, MN (US); David R. STUMP, Columbia City, IN (US); Alexander Paul WOLFE, Fort Wayne, IN (US)

(21) Appl. No.: 18/038,663

(22) PCT Filed: Feb. 11, 2022
(86) PCT No.: PCT/US2022/016106

§ 371 Date: May 24, 2023

Related U.S. Application Data
(60) Provisional application No. 63/148,532, filed on Feb. 11, 2021.

(51) Int. Cl. A61F 2/40; A61F 2/30; A61L 27/56; A61L 27/16

(52) U.S. Cl. CPC A61F 2/4081; A61F 2/30749; A61L 27/56; A61L 27/16; A61F 2/30771; A61F 2002/3093; A61F 2002/30884; A61F 2002/3092; A61F 2002/30878; A61L 2430/02; A61L 2300/412

(57) ABSTRACT

Glenoid implants, used in shoulder reconstruction or replacement surgery include a main body having an articulation surface configured to interface with the humeral head and having an anchoring surface opposite the articulation surface; and a fixation structure extending away from the anchoring surface comprising an osteogenous material portion. The fixation structures can include polymeric material, porous metallic material, porous polymer material and/or combinations thereof.

### FIELD OF DISCLOSURE

The present disclosure generally relates to glenoid implants for shoulder prosthesis.

### BACKGROUND

A shoulder prosthesis includes a glenoid implant intended to replace the glenoid cavity of the scapula and/or a humeral implant intended to replace the humeral head. The glenoid implant generally includes and articular body intended to articulate with the humeral head, and a fixation means to stabilize the articular body with respect to the scapula.

One concern during the glenoid implant process is the preservation of bone material. In certain situations, it is beneficial to maintain natural bone material, thus allowing for more natural and effective adaptation of the newly implanted devices. In addition, efficiency and effectiveness of implanted devices are also significant considerations, so patients can easily recover and quickly develop full use of their shoulder joints. In many cases, the promotion of natural tissue ingrowth will help, causing natural tissues to surround and/or adhere to the implanted devices or components. On a similar note, the minimization of cement or related adhesives during the implant process is also desired, as this will promote a more natural acceptance of implanted devices. Thus, continuous improvements in glenoid implant designs and shoulder implant systems that cooperate with these glenoid implants is desired.

### BRIEF DESCRIPTION OF THE DRAWINGS

The various embodiments of the inventive glenoid implants of the present disclosure will be described in more detail in conjunction with the following drawing figures. The structures in the drawing figures are illustrated schematically and are not intended to show actual dimensions. Further objects and advantages of the embodiments described below can be seen from reading the detailed description in conjunction with the drawings in which:

FIG. 1 is a front view (from the articulation surface (e.g. the bearing surface)) of an inlay glenoid implant illustrating one well-accepted configuration;

FIG. 2A through FIG. 2D illustrate one embodiment of the connection structure utilized for securing an inlay glenoid implant;

FIG. 3 is a rear view of yet another embodiment of an inlay glenoid implant;

FIGS. 4A through 4C presents a further embodiment of an inlay glenoid implant;

FIGS. 5A through 5C illustrate one embodiment of an onlay glenoid implant;

FIGS. 6A through 6C show another embodiment of an onlay glenoid implant;

FIGS. 7A and 7B present a further embodiment of an onlay glenoid implant;

FIGS. 8A and 8B show an additional embodiment of an onlay glenoid implant;

FIGS. 9A and 9B illustrate yet another embodiment of an onlay glenoid implant design;

FIGS. 10A and 10B depicts an additional embodiment of an onlay glenoid implant;

FIGS. 11A and 11B present another alternative embodiment of an onlay glenoid implant; and

FIGS. 12A and 12B illustrate a further embodiment of an onlay glenoid implant.

FIGS. 13A and 13B illustrate a further embodiment of an onlay glenoid implant having a composite fixation structure.

FIGS. 14A and 14B illustrate a further embodiment of an onlay glenoid implant having a composite fixation structure.

FIGS. 15A and 15B illustrate a further embodiment of an inlay glenoid implant using a composite fixation structure.

FIGS. 16A and 16B illustrate a further embodiment of an inlay glenoid implant using a composite fixation structure.

FIGS. 17A and 17B illustrate a further embodiment of an onlay glenoid implant.

### DESCRIPTION

Glenoid implants, used in shoulder reconstruction or replacement surgery, are well-recognized components and generally follow two different design approaches; an onlay configuration where the glenoid implant is placed over the glenoid bone structure, and an inlay configuration where the glenoid implant structure is recessed within a portion of the glenoid bone structure itself. In addition, glenoid implants (both onlay and inlay) typically have some connecting structure that is designed to be inserted into the bone structure and provide the necessary connection. Ideally, these connection mechanisms include features that will accommodate some amount of bone cement, while also having features that promote tissue ingrowth and natural acceptance of the implant into the human body. Typically, the use of bone cement is minimized, and natural tissue growth is promoted. That said, there is a balance between these two features, as removal may become necessary.

As mentioned above, glenoid implants can utilize an inlay or an onlay configuration. The inlay approach requires the disruption of more bone structure in the scapula, but does leave some bone structure in place around the periphery of a recess created in the glenoid to receive the inlay glenoid implant. Once the inlay glenoid implant is placed within the cavity, the bearing surface of the implant itself forms a portion of the glenoid cavity along with the bone structure around the periphery of the glenoid cavity. Consequently, the dimensions of the implant main body and the connection structure must be carefully considered. With an onlay approach, the implant will sit on top of the glenoid bone structure, and will have a surface area which creates the entire “new” glenoid cavity. Generally, onlay designs can be sized larger, as they are not required to be inset into bone structure. That said, a connection structure is required to retain the onlay glenoid implant in place.

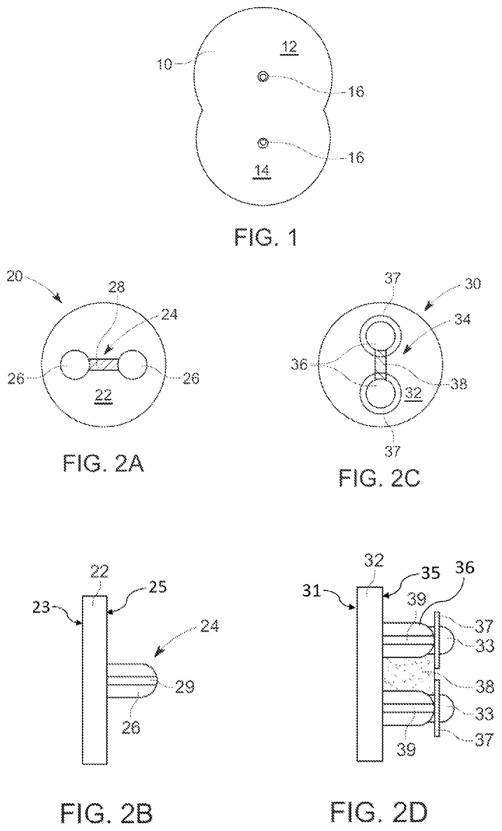

Referring now to FIG. 1, a front view of an exemplary inlay design glenoid implant 10 is presented. As can be seen, as shown, this front view of the glenoid inlay implant 10 shows an irregular shaped main body having a first circular portion 12 and a second circular portion 14 which are both integrally connected to one another to form a continuous surface. According to some embodiments, the glenoid implant 10 can include a pair of drill guides 16 for use in contemplated drilling procedures. Although not specifically illustrated, a depth stop device can be utilized to control the depth of drilling during operations. Throughout this disclosure, front of a glenoid implant refers to the articulation surface (e.g. the bearing surface) side of the implant and rear or back of the glenoid implant refers to the anchoring surface that is generally on the opposite side of the front. The anchoring surface is configured with one or more of various fixation structures to engage the glenoid bone and secure the glenoid implant to the glenoid when implanted.

As an inlay implant, the glenoid implant 10 will be inset or implanted into a recess in the bone rather than on top of it. This approach preserves a portion of the natural bone surrounding the glenoid implant 10, thereby promoting a more natural joint function. The specific configuration illustrated in FIG. 1 shows one example which could be used, although other shapes are equally possible including ovals or circular structures. It will also be recognized and understood that one or more fixation or attachment structures (also known as anchoring structures) are provided on the back side of the inlay glenoid implant 10 (not shown in FIG. 1). According to the present disclosure, many different fixation structures can be utilized which are discussed and outlined in various detail below. Onlay glenoid implants are also provided on their back side with one or more fixation structures. For both inlay and onlay glenoid implants, the patient's glenoid are prepared with appropriate hole(s) or recess(es) to receive the one or more fixation structures.

Disclosed herein are various embodiments for improved configurations for the fixation structures to enhance the anchoring of the glenoid implants to the patient's glenoid. The improved configurations for fixation structures disclosed herein are designed to minimize or eliminate the undesired micromotion of the implanted glenoid implant by enhancing quality of the anchoring to the glenoid. The disclosed embodiments of the improved fixation structures can be implemented into inlay or onlay glenoid implants regardless of whether the particular example for an improved fixation structure is disclosed herein with an inlay configuration glenoid implant body or an onlay configuration glenoid implant body.

With various designs for inlay glenoid implants, the main body of the glenoid implant will have a depth which is closely controlled to avoid excess disruption of bone structure. In the implant procedures, concerns exist related to the violation of the subchondral plate, and unnecessary violation of the cancellous vault. In certain patients, the bone structure of the glenoid vault can vary; however, it is somewhat common to encounter less than 1.5 mm of calcified cartilage, and the subchondral plate having a subsequent thickness of 2 to 4.5 mm. This can vary depending on the patient and their condition must be taken into consideration when configuring an inlay glenoid implant.

Referring to FIGS. 2A through 2D, a first design concept is illustrated for an inlay glenoid implant fixation structure. FIG. 2A illustrates a rear view of an embodiment of a glenoid implant 20. FIG. 2B illustrates a side view, and further illustrates certain details of a fixation structure 24. In this embodiment, the glenoid implant 20 includes a main body 22 having an articulation surface 23 and a back surface 25 on the opposite side thereof. The main body 22 can have a predetermined thickness to accommodate the bone structure concerns mentioned above. Although the illustrated example of the implant 20 is shown with the main body 22 having a circular outline but the body of the glenoid implants disclosed herein can have any appropriate outline shape.

The fixation structure 24 can include two or more anchoring posts 26 and crossbar segment(s) 28 extending across the space between any two adjacent posts 26. In the illustrated example shown in FIG. 2A, two posts 26 are shown but three or four such posts 26, for example, can be provided. When more than two anchoring posts 26 are provided, they can be arranged in a linear arrangement if the space allows or the posts 26 can be provided in non-linear arrangement. For example, three anchoring posts can be arranged in a triangle configuration. For implanting the glenoid implant 20, the glenoid cavity would be appropriately prepared with holes or recesses to accommodate the fixation structure 24.

In this embodiment, main body 22 and posts 26 are formed of a nonmetallic material, generally a polymer or plastic compound made up of well-recognized medically acceptable materials, such as ultra-high-molecular-weight polyethylene (UHMWPE), polyether ether ketone (PEEK), etc. Further, the crossbar segment(s) 28 can be formed from an osteogenous material such as a porous metallic material or porous UHMWPE, which is specifically selected to promote natural bone tissue ingrowth to enhance bonding/anchoring of the glenoid implant to glenoid after the implant is implanted. An example is the porous trabecular metallic material such as Wright Medical Technology's ADAPTIS™. In some embodiments, the crossbar segment(s) 28 can be coated with the osteogenous material. In some embodiments, the crossbar segment(s) 28 can be entirely formed of the osteogenous material or some portion of the crossbar segment(s) 28 can be formed of the osteogenous material.

As illustrated in the side view shown in FIG. 2B, each anchoring post 26 can include one or more channels 29 on the outer surface of the post. extending the length of the post on an outer side thereof. In use, the channels 29 provide the space to accommodate bone cement, as necessary. Each of the channels 29 can be provided as extending longitudinally along the length of the post 26 as shown in FIG. 2B. In some embodiments, the one or more channels 29 can be oriented at an angle with respect to the longitudinal axis of the posts 26. For example, the one or more channels 29 on a given post can be oriented orthogonal to the longitudinal axis of the post 26.

FIGS. 2C and 2D illustrate a similar design. More specifically, FIG. 2C shows a glenoid implant 30 comprising a main body 32 including an articulation surface 31, a back surface 35, and a fixation structure 34 extending from the back surface. The fixation structure 34 can include two or more anchoring posts 36 and crossbar segment(s) or wall(s) 38 formed of a porous metallic material to promote ingrowth of bone tissue. In the illustrated example shown in FIGS. 2C and 2D, two anchoring posts 36 are shown but three or four such posts 36, for example, can be provided. The geometric arrangement of these posts 36 can be varied as mentioned previously for the posts 26 in the implant embodiment 20. In this embodiment, one or more fins 37 are provided on each post 36 to help hold implant 30 in place on the glenoid when implanted. In the illustrated example shown in FIG. 2D, one fin 37 is provided on each post 36. Each fin 37 radially extend away from the post 36 and can circumscribe the post 36. Such fins 37 are generally made of the same polymer material as the posts 36 and are dimensioned to be resiliently flexible. The hole or a recess in the glenoid for receiving the post 36 would be dimensioned to have a slightly smaller diameter than the fins 37 so that when the post 36 is inserted into the prepared recess, a press-fitting engagement is achieved between the flexible fins 37 and the inner sidewall of the prepared recess. In some embodiments, the crossbar segment(s) 38 can be coated with the osteogenous material. In some embodiments, the crossbar segment(s) 38 can be entirely formed of the osteogenous material or some portion of the crossbar segment(s) 38 can be formed of the osteogenous material.

As shown in FIG. 2D, each post can also be configured with one or more channels 39 configured to accommodate bone cement. In the embodiment shown in FIG. 2D, each of the posts 36 has an extended section 33 extending from a top portion thereof to help support or accommodate the one or more fins 37. In some other embodiments, the one or more fins 37 can extend directly from the posts 36 without the need for the extended section 33. In such embodiments, the one or more channels 39 can cut through the fins 37 as necessary depending on the particular orientation of the one or more channels 39 provided.

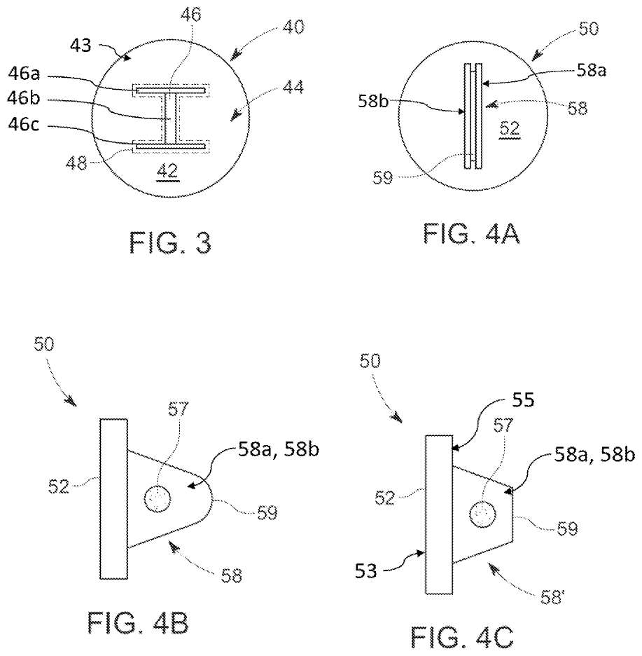

FIG. 3 illustrates yet another embodiment of an inlay glenoid implant 40 which uses an alternative fixation structure 44. More specifically, the fixation structure 44 includes an I-beam structure 46 extending from a back surface 43 of main body 42. The I-beam structure 46 comprises three segments 46a, 46b, and 46c that form the I-beam shape. The glenoid would be prepared with a recess that has a corresponding I-beam shape to receive the fixation structure 44. The I-beam structure can provide enhanced torsional resistance when the glenoid implant 40 is implanted in a glenoid.

To further enhance bone tissue ingrowth, the I-beam structure 46 can be coated with a porous metallic material 48. The porous metallic material 48 can cover substantially the entire surface of the I-beam structure 46 or one or more portions of the surface of the I-beam structure 46. As used herein, “substantially the entire surface” refers to at least 80% of the involved surface. The three segments 46a, 46b, and 46c of the I-beam structure 46 can extend from the back surface 43 equal distance so that the three segments have the same height and extend into the receiving glenoid a predetermined distance when implanted. In some embodiments, the three segments 46a, 46b, and 46c can each have different height to accommodate different conditions or quality of the receiving glenoid. In some embodiments, the distal ends (the ends that are away from the back surface 43) of the three segments 46a, 46b, and 46c can have varying height within each segment.

In some embodiments, rather than being coated with the porous metallic material 48, one or more of the three segments 46a, 46b, and 46c of the I-beam structure 46 can be made of the porous metallic material in whole. In some embodiments, one or more of the three segments 46a, 46b, and 46c of the I-beam structure 46 can be made of a porous UHMWPE material. Such porous UHMWPE also can promote bone tissue ingrowth.

FIGS. 4A through 4C illustrates a further embodiment of an inlay glenoid implant 50. The implant 50 comprises a main body 52 having an articulation surface 53, and a back surface 55. The illustrated example of the main body 52 is shown in a circular format, however, could be shaped in alternative configurations to meet the necessary purpose or application. In this embodiment a keel design is utilized for the fixation structure, with a keel structure 58 including a central channel 59 extending along a length of the keel structure 58. The channel 59 is designed and configured to hold or accommodate some amount of bone cement during the implantation procedure. The keel structure 58 is a generally flat structure extending from the back surface 55 and has two major flat surfaces 58a and 58b as shown in FIG. 4A. When viewed from the side, looking at one of the two major surfaces as shown in FIGS. 4B and 4C, the keel structure 58 is configured to have a shape that is wider at the end joining the back surface 55 and narrower at the distal end. In the example shown in FIG. 4B, the keel structure 58 has a triangular shape with a rounded distal tip. In the example shown in FIG. 4C, the keel structure 58′ has a trapezoid shape with the end distal from the back surface 55 being shorter than the end joining the back surface 55. Further, the keel structure 58 can be formed to include a porous scaffold material portion 57 that can promote bone tissue ingrowth in this area. The porous scaffold material portion 57 can be made of a porous metallic material or porous UHMWPE material. The porous scaffold material portion 57 can extend through the thickness of the keel structure 58, 58′ from one major surface 58a to the other major surface 58b thus presenting a porous scaffold material portion on each of the major surfaces 58a, 58b as shown in FIGS. 4B and 4C to promote bone tissue ingrowth after the implant 50 has been implanted into a patient.

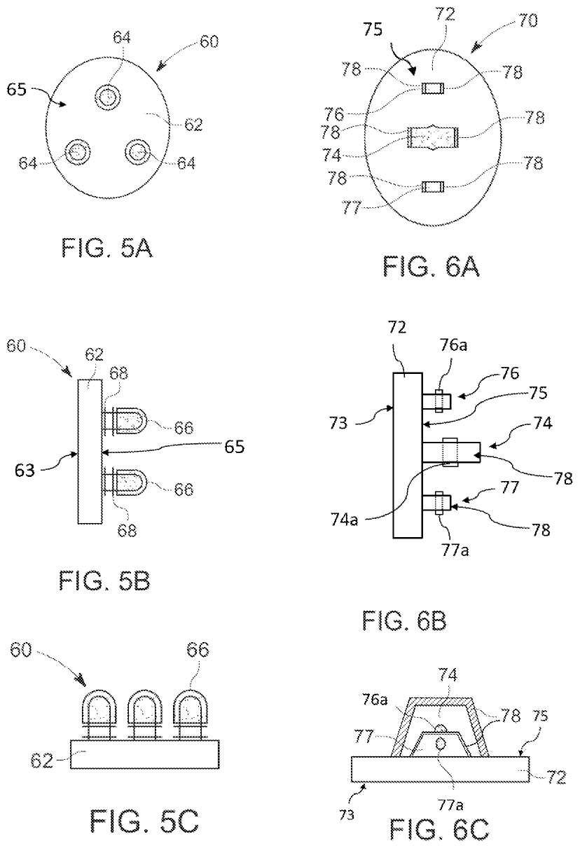

According to some embodiments, FIGS. 5A through 5C illustrate a glenoid implant 60. This particular embodiment is designed to be used in an onlay application, thus is sized and configured to cover the related bone structure (as opposed to being inset or inlaid therein). As illustrated, the glenoid implant 60 comprises a main body 62 having an articulation surface 63 and back surface 65 configured in a substantially ovoid disc-shape structure. Further illustrated is a fixation structure configuration that includes one or more anchoring posts 64, with each post 64 having a cap 66 and one or more optional fins 68 provided between the cap 66 and the back surface 65. In the illustrated example, three anchoring posts 64 are shown. As shown, FIG. 5A presents a rear view of the glenoid implant 60, while FIG. 5B illustrates a side view and FIG. 5C illustrates a different side view from a different viewing angle.

In this embodiment, the main body 62 and the posts 64 can be formed from a medical grade polymer or plastic material discussed above, while caps 66 are porous metallic material promoting bone tissue ingrowth. It will be recognized that caps 66 could be replaced by porous metallic cylinders (not shown) that surround posts 64. In some embodiments, the porous metallic material can be provided as a coating and cover only a portion or the full length of the posts 64. Additionally, radially extending fins 68 positioned between the caps 66 and the main body 62 are utilized to provide further security of the implant 60 to a patient.

FIGS. 6A through 6C illustrate yet another embodiment of an onlay glenoid implant 70, which incorporates a plurality of keels for the fixation structures. The glenoid implant embodiment 70 comprises a main body 72 having an articulation surface 73 and a back surface 75 and three keels, a central keel 74, a first side keel 76, and a second side keel 77, extending from the back surface 75. As illustrated, the main central keel 74 extends from the back surface 75 of the main body 72 at a central portion thereof. Flanking the central keel 74 are the first side keel 76 and the second side keel 77. The three keels can be all of same size or can be configured to have different sizes. In the illustrated example, the central keel 74 is larger than the two side keels 76, 77. The main body 72 can have an oval or a kidney shaped outline to match the shape of the glenoid. When implanted, such glenoid implant 70 would generally be oriented so that the three keels align in superior to inferior anatomical direction.

According to the present disclosure, the keels 74, 76, and 77 can incorporate porous metallic material at various portions of the keels to promote bone tissue ingrowth. As shown in the view of the back surface 75 in FIG. 6A and the side view from the second side keel 77 end of the main body 72 in FIG. 6C, the outer surface portions 78 along the edges of one or more of the keels 74, 76, and 77 can be coated with or made of the porous metallic material. Additionally, as shown in FIG. 6C and the side view along the edges of the keels shown in FIG. 6B, each of the keels 74, 76, and 77 can also have one or more portions extending from one side surface of the keel to the opposite side surface of the keel that is formed of the porous metallic material. One such portion is illustrated as an example for each of the three keels 74, 76, and 77 and they are identified as 74a, 76a, and 77a, respectively, in FIG. 6B. FIG. 6B is a side view from an angle orthogonal to the view taken in FIG. 6C.

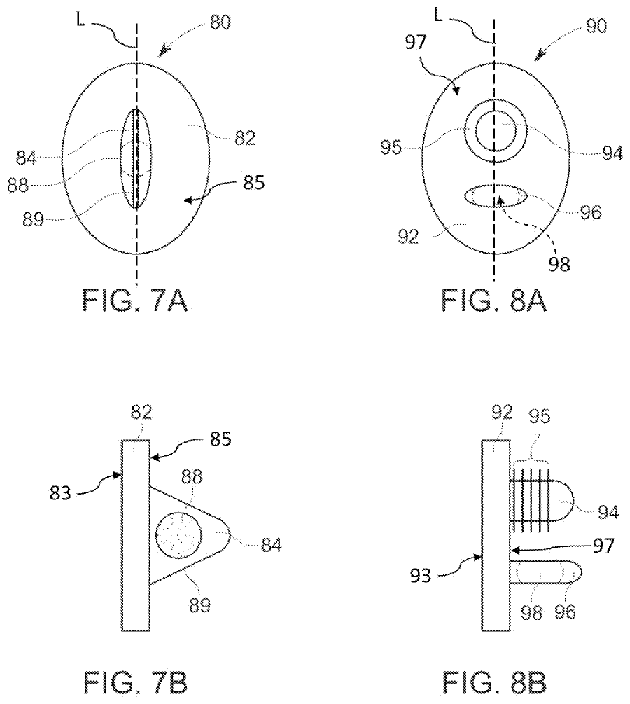

Turning now to FIGS. 7A and 7B, a further embodiment of an onlay glenoid implant 80 is shown. The glenoid implant 80 comprises a main body 82 having an articulation surface 83 and a back surface 85. In this embodiment, glenoid implant 80 utilizes a central keel structure 84 for a fixation structure. In this design, central keel structure 84 includes a central porous metallic section 88 extending through the keel structure 84, which again is utilized to promote bone tissue ingrowth. In addition, the keel 84 can include an edge channel 89 which is positioned and configured to accommodate some amount of bone cement when the glenoid implant 80 is implanted. In this embodiment, the keel structure 84 is oriented in line with the line L representing superior-inferior direction when the glenoid implant 80 is implanted in a glenoid. As shown in FIG. 7A, the keel 84 can have curved surfaces and present more rounded profile that can improve insertion into a prepared recess in a glenoid.

FIGS. 8A and 8B present an additional embodiment of a glenoid implant 90. In this embodiment, glenoid implant 90 includes a main body 92 having an articulation surface 93 and a back surface 97 that incorporates an anchoring post and a keel fixation structure. More specifically, the anchoring post 94 extends from the back surface 97 of the main body 92, with the post 94 also including one or more fins 95. The glenoid implant 90 also includes a keel structure 96 extending from the back surface 97 of main body 92. In this embodiment, the keel structure 96 is oriented orthogonal to the line L representing superior-inferior direction when the glenoid implant 90 is implanted in a glenoid. As shown in FIG. 8B, keel 96 can include a portion of porous metallic material 98 to promote bone tissue ingrowth.

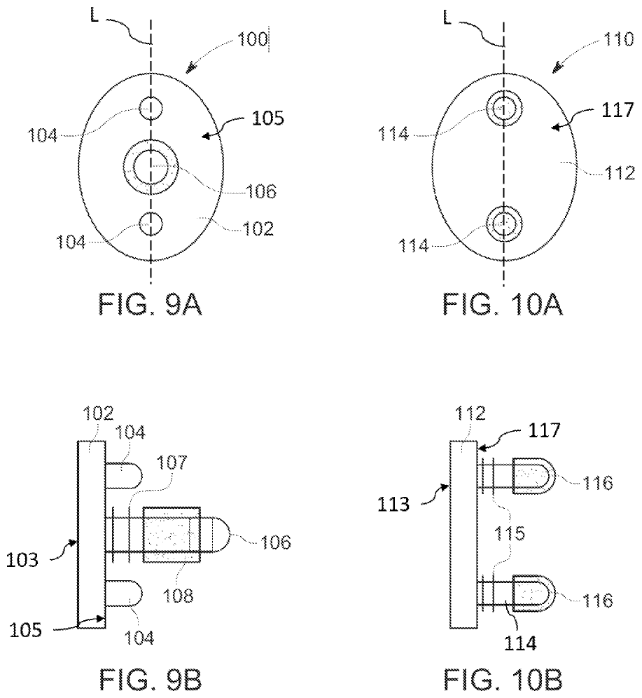

FIGS. 9A and 9B shows another embodiment of an onlay glenoid implant 100 which makes use of a triple anchoring post fixation structure. The glenoid implant 100 comprises a main body 102 having an articulation surface 103 and a back surface 105. Here, a pair of pegs 104 extend from the back surface 105 of the main body 102 at a superior position and an inferior position. In a similar manner, a central anchoring post 106 extends outwardly from the back surface 105 of the main body 102 at a central location. In some embodiments, the pegs 104 and the central post 106 are linearly aligned in superior-inferior direction. FIG. 9A shows a line L representing the superior-inferior direction when the glenoid implant 100 is implanted in a glenoid. The terms pegs and posts both refer to generally cylindrical shaped fixation structures extending from the back surface but pegs refer to structures that are smaller than posts in a given glenoid implant as with the pegs 104 and the post 106. The central post 106 can include one or more fins 107 to help stabilize glenoid implant 100 when placed in a patient. In addition, the central post 106 can also include a porous metallic material cylinder 108 surrounding a portion of the central post 106 to enhance anchoring into a glenoid by promoting bone tissue ingrowth.

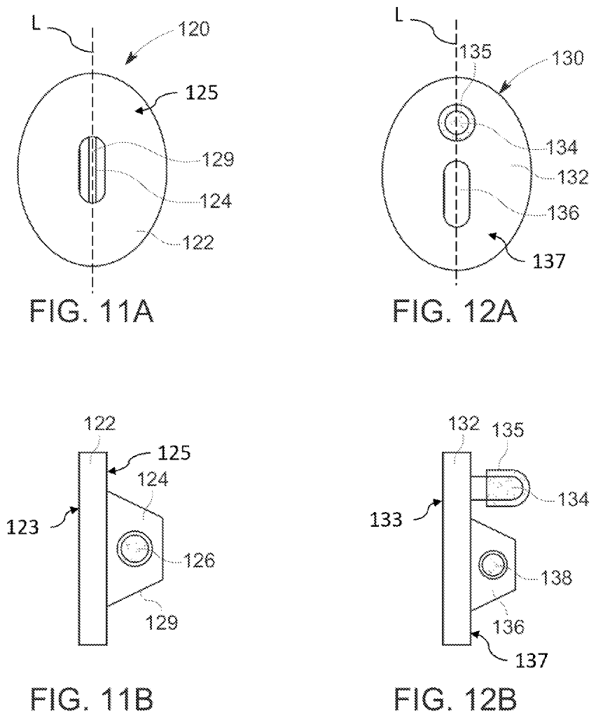

Illustrated in FIGS. 10A and 10B is a further embodiment of an onlay glenoid implant 110 which makes use of a dual post fixation structure. The glenoid implant 110 comprises a main body 112 having an articulation surface 113 and a back surface 117. Here, a pair of posts 114 extend from the back surface 117 of the main body 112 at a superior position and an inferior position. In some embodiments, the posts 114 are aligned along the superior-inferior direction represented by the line L in FIG. 10A. As best illustrated in FIG. 10B, each of the posts 114 can include one or more fins 115, and a porous metallic cap 116. Although not specifically illustrated, it is also contemplated that this embodiment could utilize porous metallic cylinders (similar to that illustrated in FIG. 9B above) which would perform a function similar to the porous metallic caps 116, however the porous metallic cylinders would simply surround the side portions of posts 114 only without completely covering the tip portions of the posts.

Turning now to FIGS. 11A and 11B, a keel design is illustrated for another embodiment of an onlay glenoid implant 120. The glenoid implant 120 comprises a main body 122 having an articulation surface 123 and a back surface 125. More specifically, a keel structure 124 extends outwardly from the back surface 125 of the main body 122. Similar to the keels 74, 76, and 77 in the glenoid implant embodiment 70, the keel 84 in the glenoid implant embodiment 80, and the keel 96 in the glenoid implant embodiment 90, the keel 124 can include a porous metallic material portion 126 that extends through the thickness of the keel 124 to promote bone tissue ingrowth. As shown in FIG. 11B, the porous metallic material portion 126 forms a portion of each of the two major side surfaces of the keel 124. Also included along an edge of keel 124 is a channel 129 that can accommodate bone cement use during the implant procedure. The keel 124 is oriented along the superior-inferior direction of the glenoid implant 120 represented by the line L in FIG. 11A. Superior-inferior direction refers to the anatomical direction when the glenoid implant 120 is implanted in a glenoid.

A further embodiment of an onlay glenoid implant 130 is presented in FIGS. 12A and 12B. The glenoid implant 130 comprises a main body 132 having an articulation surface 133 and a back surface 137. As can be seen, this design utilizes a post 134 and a keel structure 136 for the fixation structures. In some embodiments, the post 134 and the keel structure 136 are arranged along the superior-inferior direction represented by the line L in FIG. 12A. The keel 136 is oriented along the superior-inferior direction of the glenoid implant 130 represented by the line L. In some embodiments, the glenoid implant 130 can be implanted in a glenoid so that the post 134 is positioned superior to the keel 136 when implanted. In some embodiments, the glenoid implant 130 can be implanted in a glenoid so that the post 134 is positioned inferior to the keel 136 when implanted. The post 134 extends outwardly from the back surface 137 of the main body 132, at an upper location. A metal cap 135 can be configured on top of the post 134 Similar to the keel 124 of glenoid implant 120, the keel 136 can include a porous metallic material portion 138 that extends through the thickness of the keel 136 to promote bone tissue ingrowth. In an additional embodiment, the keel 136 can include a channel to accommodate bone cement therein.

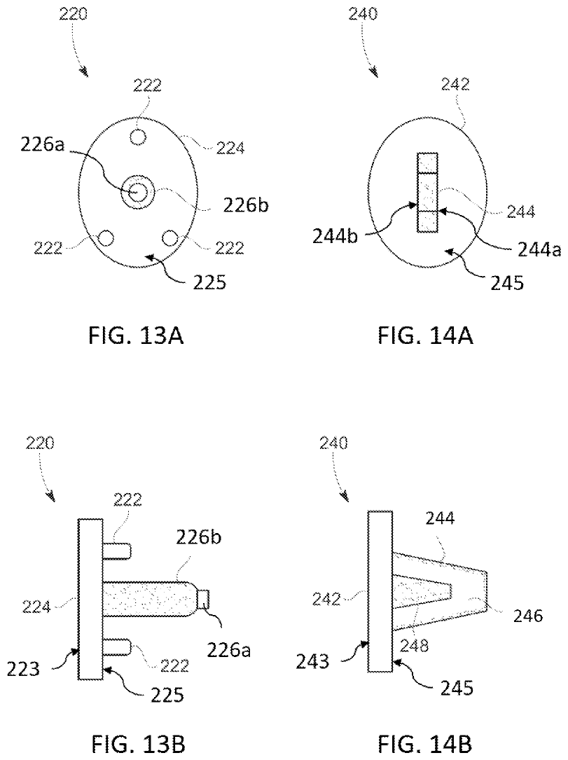

FIGS. 13A and 13B show another embodiment of an onlay glenoid implant 220 which makes use of a hybrid or composite fixation structure 226. The glenoid implant 220 comprises a main body 224 having an articulation surface 223 and a back surface 225. Here, a plurality of pegs 222 extend from the back surface 225 of the main body 224 and are positioned about the main body 224. In the illustrated embodiment, three pegs are illustrated, although any number of pegs can be used. A central post 226 extends outwardly from the back surface 225 of the main body 224 at a central location. The central post 226 has a composite structure comprised of a porous UHMWPE core 226a surrounded by a porous metallic material shell 226b both of which promote bone tissue ingrowth. This composite structure provides the central post 226 that is wholly formed of osteogenous material, which promotes attachment and desired tissue ingrowth. The porous metallic material shell 226b provides some structural rigidity to the central post 226.

Turning now to FIGS. 14A and 14B, a keel design is illustrated for another embodiment of an onlay glenoid implant 240. The glenoid implant 240 comprises a main body 242 having an articulation surface 243 and a back surface 245. A keel structure 244 having a composite structure extends outwardly from the back surface of the main body 242. The composite structure of the keel 244 comprises a porous UHMWPE core portion 248 surrounded by an outer edge portion 246 that is made of a porous metallic material. The porous UHMWPE core portion 248 extends through the full thickness of the keel 244 and presents an exposed porous UHMWPE portion on the two major surfaces 244a, 244b of the keel 244. In some embodiments, the outer edge portion 246 is made of a porous metallic material.

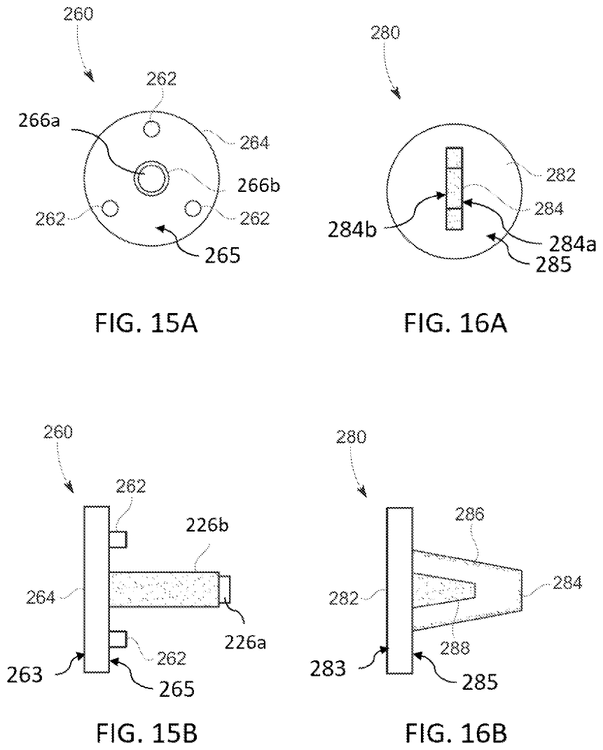

FIGS. 15A and 15B shows an embodiment of an inlay glenoid implant 260 which makes use of a hybrid or composite fixation structure 266. The glenoid implant 260 comprises a main body 264 having an articulation surface 263 and a back surface 265. Here, a plurality of pegs 262 extend from the back surface 265 of the main body 264 and are positioned about the main body 264. In the illustrated embodiment, three pegs are illustrated, although any number of pegs can be used. A central post 266 extends outwardly from the back surface 265 of the main body 264 at a central location. The central post 266 has a composite structure comprised of a porous UHMWPE core 266a surrounded by a porous metallic material shell 266b both of which promote bone tissue ingrowth. This composite structure provides the central post 266 that is wholly formed of osteogenous material, which promotes attachment and desired tissue ingrowth. The porous metal material shell 266b provides some structural rigidity to the central post 266.

Turning now to FIGS. 16A and 16B, a keel design is illustrated for another embodiment of an inlay glenoid implant 280. The glenoid implant 280 comprises a main body 282 having an articulation surface 283 and a back surface 285. A keel structure 284 having a composite structure extends outwardly from the back surface 285 of the main body 282. The composite structure of the keel 284 comprises a porous UHMWPE core portion 288 surrounded by an outer edge portion 286 that is made of a porous metallic material. The porous UHMWPE core portion 288 extends through the full thickness of the keel 284 and presents an exposed porous UHMWPE portion on the two major surfaces 284a, 284b of the keel 284. In some embodiments, the outer edge portion 286 is made of a porous metallic material.

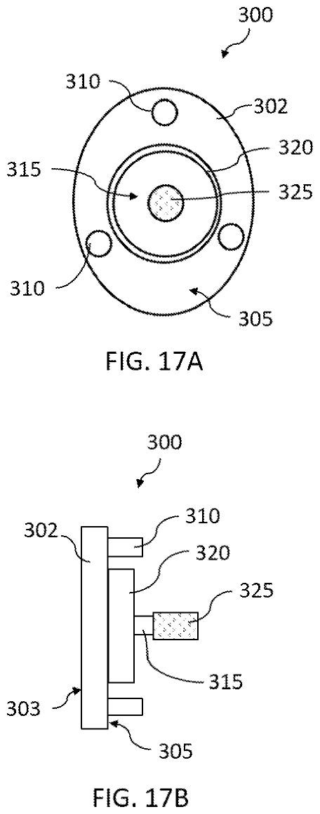

FIGS. 17A and 17B illustrate another example of a glenoid implant 300 incorporating improved fixation structures. The glenoid implant 300 comprises a main body 302 having an articulation surface 303 and a back surface 305. For a fixation structure, the glenoid implant 300 can be configured with a hybrid anchor post 315 extending from the back surface 305 for engaging the prepared surface of the glenoid and anchor the glenoid implant 300. The hybrid anchor post 315 includes a modular osteogenous portion 325 formed of an osteogenous material that provides enhanced securement between the anchor post 315 and the glenoid. The osteogenous portion 325 can be formed of a porous metallic material or a porous UHMWPE. The modular porous metallic material portion 325 can be configured to be removably attached to the anchor post 315, such as by screw thread structures. For example, the distal tip of the anchor post 315 can be provided with male screw threads and the porous metallic material portion 325 can be configured with corresponding female screw threads. In some embodiments, the male/female assignment of the screw threads on the two components can be reversed. Because this configuration is modular, the porous metallic material portion 325 can be provided in a variety of shapes and sizes to accommodate different patient bone quality and condition.

In some embodiments, the glenoid implant 300 can further comprise a stabilization ring structure 320 that extends from the back surface 305. The stabilization ring 320 extends from the back surface 305 to some distance so that the ring 320 is actually a short cylindrical structure. When the glenoid implant 300 is implanted into a glenoid, the ring 320 extends into the glenoid providing an anchoring engagement that spans 360 degrees angle. In some embodiments, the ring structure 320 is made of the same polymer material as the main body 302. The stabilization ring 320 enhances the overall quality of the anchoring of the glenoid implant 300 to the glenoid to reduce, minimize, or prevent micromotion of the implant after the glenoid implant is implanted in a patient.

Similar to the dimensions of the various pegs, anchoring posts, and keels described herein in connection with the various embodiments of the glenoid implants, the dimensions of the ring 320, such as the height, thickness, diameter, etc., is to be optimized depending on the size, condition, and quality of the glenoid of the patient.

In the illustrated example, the anchor post 315 extends from the back surface 305 at a position that is inside the ring 320. In some embodiments, the anchor post 315 extends from the back surface from a position that is at the geometric center of the ring 320 but the present invention is not limited as such. The position of the anchor post 315 can be off-center with respect to the stabilization ring 320. In some embodiments, the outline shape of the stabilization ring 320 is not circular but oval. In some embodiments, the outline shape can have a complex irregular curvature if it is to be matched to the particular shape of the patient's glenoid shape.

In some embodiments, the glenoid implant 300 can further comprise a plurality of additional anchoring posts 310 that extends from the back surface 305 that are located outside the ring 320. In the illustrated example, three such anchoring posts 310 are provided. Each of the anchoring posts 310 can be configured with one or more channels to accommodate bone cement. Each of the anchoring posts 310 can be configured with one or more fins similar to the fins 95 provided on the posts 94 of the glenoid implant 90.

In some embodiments, the porous metallic structures described in connection with any of the glenoid implant embodiments can be formed of porous UHMWPE material.

Various embodiments of glenoid implants have been described above in detail. It is understood and recognized that various combinations of features are possible, several of which include various advantages. Further, it will be recognized that implant procedures could vary depending on the type of fixation structure used. For example, the use certain fixation structures which have smaller extensions could better support the use of low energy drilling/reaming and punch like devices to minimize thermal damage to the patient's bone structure. Overall, this would also allow for the preservation of more bone structure.

Various embodiments of the invention have been described above for purposes of illustrating the details thereof and to enable one of ordinary skill in the art to make and use the invention. The details and features of the disclosed embodiment[s] are not intended to be limiting, as many variations and modifications will be readily apparent to those of skill in the art. Accordingly, the scope of the present disclosure is intended to be interpreted broadly and to include all variations and modifications coming within the scope and spirit of the appended claims and their legal equivalents.

1. A glenoid implant comprising:
a main body having an articulation surface configured to interface with the humeral head and having an anchoring surface opposite the articulation surface; and
a fixation structure extending away from the anchoring surface comprising an osteogenous material portion.

2. The glenoid implant of claim 1, wherein the osteogenous material portion comprises a porous metallic material and/or a porous ultra-high-molecular-weight-polyethylene.

3. The glenoid implant of claim 1, wherein the fixation structure is further configured to accommodate an amount of bone cement.

4. The glenoid implant of claim 1, wherein the fixation structure is an I-beam extension and the osteogenous material portion covers substantially the entire outer surface of the I-beam extension, wherein the I-beam extension has an end surface opposite the main body and the cement application surface is positioned on the end surface.

5. The glenoid implant of claim 1, wherein the fixation structure comprises one or more anchoring posts extending from the anchoring surface, and wherein each anchoring post has the osteogenous material provided on a portion of an outer surface of the anchoring post.

6. The glenoid implant of claim 5, wherein the glenoid implant comprises two or more anchoring posts and further comprising a crossbar segment extending between a pair of the two or more anchoring posts, and wherein the crossbar segment comprises the osteogenous material.

7. The glenoid implant of claim 6, wherein the crossbar segment is coated with a porous metallic material.

8. The glenoid implant of claim 6, wherein the crossbar segment comprises a porous metallic material or a porous ultra-high-molecular-weight-polyethylene.

9. The glenoid implant of claim 5, wherein a portion of one or more of the anchoring posts is configured to accommodate bone cement.

10. The glenoid implant of claim 5, wherein at least one of the one or more anchoring posts is configured with fins.

11. The glenoid implant of claim 1, wherein the fixation structure includes at least one keel.

12. The glenoid implant of claim 11, wherein the osteogenous material portion comprises a porous metallic material and/or a porous ultra-high-molecular-weight-polyethylene.

13. The glenoid implant of claim 12, wherein the osteogenous material portion extends through full thickness of the at least one keel.

14. The glenoid implant of claim 12, wherein the osteogenous material portion includes an edge of the at least one keel.

15. The glenoid implant of claim 11, wherein the at least one keel is further configured to accommodate an amount of bone cement.

16. A glenoid implant comprising:
a main body having an articulation surface configured to interface with the humeral head and having an anchoring surface opposite the articulation surface; and
a fixation structure extending away from the anchoring surface comprising an osteogenous material portion that includes a composite structure of two different osteogenous materials.

17. The glenoid implant of claim 16, wherein the two osteogenous materials are a porous metallic material and a porous ultra-high-molecular-weight-polyethylene.

18. The glenoid implant of claim 16, wherein the fixation structure comprises a core formed of porous ultra-high-molecular-weight-polyethylene surrounded by a region of porous metallic material.

19. A glenoid implant comprising:
a main body having an articulation surface configured to interface with the humeral head and having an anchoring surface opposite the articulation surface; and
a fixation structure extending away from the anchoring surface comprising an osteogenous material portion that includes a modular portion of an osteogenous material.

20. The glenoid implant of claim 19, wherein the osteogenous material is a porous metallic material or a porous ultra-high-molecular-weight polyethylene.

21. The glenoid implant of claim 19, wherein the fixation structure comprises an anchor post and the modular portion of an osteogenous material is configured to be removably attached to the anchor post.

22. The glenoid implant of claim 19, wherein the fixation structure further comprises a stabilization ring structure extending from the anchoring surface.

23. The glenoid implant of claim 21, wherein the fixation structure further comprises a stabilization ring structure extending from the anchoring surface and the anchoring post extends from the anchoring surface at a position that inside the stabilization ring.

24. The glenoid implant of claim 23, wherein the anchoring post extends from the anchoring surface at a position that is at a geometric center of the stabilization ring.
